(19) United States
(12) Patent Application Publication
(10) Pub. No.: US 2026/0207447 A1
(43) Pub. Date: Jul. 23, 2026

(54) COMPOSITION WITH CHITOSAN

(71) Applicant: L'OREAL, Paris (FR)

(72) Inventors: Christophe KUSINA, Chevilly Larue (FR); Nathalie BOILEAU, Chevilly Larue (FR); Danielle LE VERGE, Chevilly Larue (FR); Géraldine LEREBOUR, Chevilly Larue (FR); Angélina ROUDOT, Chevilly Larue (FR); Isabelle TERRISSE, Chevilly Larue (FR)

(73) Assignee: L'OREAL, Paris (FR)

(21) Appl. No.: 19/139,417

(22) PCT Filed: Dec. 19, 2023
(86) PCT No.: PCT/EP2023/086535

§ 371 Date: Jun. 16, 2025

(30) Foreign Application Priority Data
Dec. 19, 2022 (FR) 2213755

(51) Int. Cl. A61K 8/29; A61K 8/365; A61K 8/37; A61K 8/60; A61K 8/73; A61Q 1/06; A61Q 3/02

(52) U.S. Cl. CPC A61K 8/29; A61K 8/365; A61K 8/37; A61K 8/60; A61K 8/73; A61Q 1/06; A61Q 3/02; A61K 2800/43

(57) ABSTRACT

The present invention relates to a cosmetic composition comprising, in a physiologically acceptable aqueous medium:
a) at least 0.01% by weight, as a proportion of the total weight of the composition, native chitosan having a molecular weight strictly greater than 3000 daltons, this quantity being strictly less than 15% by weight,
b) at least one alpha hydroxy acid,
c) from 3% to 50% pigmentary coloring material with respect to the total weight of the composition, and
d) a non-ionic surfactant,
wherein the weight ratio between non-ionic surfactant and chitosan is between 0.02 and 10, preferably between 0.1 and 6.

It also relates to a method for making up and/or treating the skin and/or keratin appendages, wherein the composition according to the invention is applied to the skin and/or keratin appendages.

The present invention relates to a cosmetic composition comprising, in a physiologically acceptable aqueous medium:
a) at least 0.01% by weight, as a proportion of the total weight of the composition, native chitosan having a molecular weight strictly greater than 3000 daltons, this quantity being strictly less than 15% by weight,
b) at least one alpha hydroxy acid,
c) from 3% to 50% pigmentary coloring material with respect to the total weight of the composition, and
d) a non-ionic surfactant,
wherein the weight ratio between non-ionic surfactant and chitosan is between 0.02 and 10, preferably between 0.1 and 6.

It also relates to a method for making up and/or treating the skin and/or keratin appendages, wherein the composition according to the invention is applied to the skin and/or keratin appendages.

There are numerous cosmetic compositions for which it is required that the deposited film, after application to keratin materials, should last well. Mention can be made for example of lipsticks or nail varnishes. In order to obtain such a result, it is possible to combine particular raw materials, in particular film-forming agents. However, the presence of such agents for the film to last well on keratin materials may lead to desiccating compositions.

Moreover, it is often sought to obtain compositions that cover well.

The formulator therefore seeks raw materials and/or systems making it possible to obtain fluid compositions (i.e. ones that flow) the deposit of which covers well and is homogeneous and strong (i.e. that lasts well).

Moreover, the cosmetic-product formulation comprising high proportions of natural raw materials or raw materials of natural origin represents one of the new challenges for satisfying the expectations of consumers. It is therefore desirable for the compositions proposed to make it possible to keep a high proportion of natural raw materials or raw materials of natural origin.

The aim of the present invention is to propose aqueous cosmetic properties having improved makeup properties, in particular in terms of coverage and homogeneity, but also lasting well.

After application, these compositions leave a homogeneous film-forming deposit that covers well and has good resistance to wear. The films formed are adhesive and cohesive, and cover and last well. In particular, they have improved resistance to friction.

The object of the present invention is therefore a cosmetic composition, in particular for make-up and/or treatment of the skin and/or lips, in particular the lips, comprising, in a physiologically acceptable aqueous medium:
a) at least 0.01% by weight, as a proportion of the total weight of the composition, native chitosan having a molecular weight strictly greater than 3000 daltons, this quantity being strictly less than 15% by weight,
b) at least one alpha hydroxy acid,
c) from 3% to 50% pigmentary coloring material with respect to the total weight of the composition, and
d) a non-ionic surfactant,
wherein the weight ratio between non-ionic surfactant and chitosan is between 0.02 and 10, preferably between 0.1 and 6.

“Physiologically acceptable” refers to a medium compatible with keratin materials.

Another object thereof is a method for making up and/or caring for the skin and/or the keratin appendages, wherein the composition according to the invention is applied to the skin and/or the keratin appendages.

### Chitosan

The composition according to the invention comprises at least 0.01%, with respect to the total weight of the composition, at least one native chitosan having a molecular weight strictly greater than 3000 daltons (3 kDa; 1 Da=1 g/mol).

The quantity of native chitosan is also strictly less than 15% by weight with respect to the total weight of the composition.

Preferably, the native chitosan has a molecular weight greater than or equal to 10 kDa, preferably greater than or equal to 15 kDa, preferably greater than or equal to 20 kDa. Preferably, the native chitosan has a molecular weight of between 10 kDa and 2 MDa preferably between 15 kDa and 1.5 MDa, preferably between 20 kDa and 300 kDa, preferably between 20 kDa and 150 kDa.

Chitosan (or chitosane) is very widespread in nature. It is signaled only in the exoskeletons of certain insects such as termite queens and in the cell walls of a particular class of mushroom, zygomycetes.

Chitosan is obtained by the deacetylation of chitin. Chitin is a polysaccharide composed of several N-acetyl-D-glucosamine units connected together by a bond of the β (1,4) type.

The ideal chemical structure of chitosan is a chain of β-D-glucosamine monomers connected by a glycoside bond (1->4).

“Chitosan” according to the invention means any copolymer formed by N-acetyl-D-glucosamine and D-glucosamine constituent units, the degree of acetylation of which is less than 90%. Chitosan consists of glucosamine sugar units (deacetylated units) and N-acetyl-D-glucosamine units (acetylated units) connected together by bonds of the β (1,4) type and constitutes a polymer of the Poly(N-acetyl-D-glucosamine)-poly(D-glucosamine) type.

Preferably, the degree of acetylation of chitosan is less than or equal to 50%, preferably less than or equal to 35%, preferably less than or equal to 25%, preferably less than or equal to 15%.

The degree of acetylation is the percentage of acetylated units with respect to the total number of units, it can be determined by Fourier transform infrared (FTIR) spectroscopy or by titration by a strong base.

The chitosan of the invention is preferably a polysaccharide prepared from a fungal origin. It is in particular extracted and purified from safe and abundant food or biotechnological fungal sources such as Agaricus bisporus or Aspergillus niger.

The chitosan of the invention preferably comes from the mycelium of a mushroom of the Ascomyceta type and in particular Aspergillus niger and/or of a Basidiomyceta mushroom, and in particular Lentinula edodes (shiitake) and/or Agaricus bisporus. Preferably the mushroom is Aspergillus niger.

The chitosan may be of GMO origin, but is preferably of non-GMO origin.

The chitosan according to the invention is native, i.e. it is not modified. In particular it does not contain any chemical modification.

One method for preparing chitosan is the one described in the application WO03068824.

The chitosan used in the invention is preferably in powder form. It is in particular marketed by Kitozyme under the name Kiosmetine or Kionutrime.

The chitosan is preferably present in a quantity ranging from 0.01% to 14% by weight, preferably from 0.1% to 14% by weight, preferably from 0.1% to 12% by weight, preferably from 0.2% to 7% by weight, and preferably from 0.25% to 5% by weight with respect to the total weight of the composition.

### Alpha Hydroxy Acid (AHA)

The composition according to the invention comprises at least one AHA.

Alpha hydroxy acid means, according to the present invention, a carboxylic acid having at least one hydroxy function occupying an alpha position on said acid (carbon adjacent to a carboxylic acid function). This acid may be in the final composition in the form of free acid and/or in the form of one of the associated salts thereof (salts with an organic base or an alkali in particular), in particular according to the final pH imposed on the composition.

The α-hydroxy acids (alpha hydroxy acids or AHA) are for example selected from lactic acid, citric acid, methyllactic acid, glucuronic acid, glycolic acid, pyruvic acid, 2-hydroxy-butanoic acid, 2-hydroxypentanoic acid, 2-hydroxyhexanoic acid, 2-hydroxyheptanoic acid, 2-hydroxyoctanoic acid, 2-hydroxy-nonanoic acid, 2-hydroxydecanoic acid, 2-hydroxyundecanoic acid, 2-hydroxydodecanoic acid, 2-hydroxytetradecanoic acid, 2-hydroxy-hexadecanoic acid, 2-hydroxyoctadecanoic acid, 2-hydroxytetra-cosanoic acid, 2-hydroxyeicosanoic acid; mandelic acid; phenyllactic acid; gluconic acid; galacturonic acid; aleuritic acid; ribonic acid; tartronic acid; tartaric acid; malic acid; fumaric acid; salts and mixtures thereof.

According to a preferred embodiment, the alpha hydroxy acid is selected from lactic acid, citric acid, malic acid, tartaric acid and salts thereof. More particularly, the alpha hydroxy acid is selected from lactic acid, citric acid and salts and mixtures thereof.

The alpha hydroxy acid or acids can be present in a quantity ranging from 0.001 to 10% by weight, from 0.005 to 5% by weight, preferably from 0.01% to 3% by weight, with respect to the total weight of the composition.

### Pigmentary Dyes

The composition according to the invention comprises from 3% to 50% at least one pigmentary dye with respect to the total weight of the composition. This dye is thus selected from powder dyes such as mineral pigments, nacres and organic pigments.

A pigmentary dye is a compound that has a color index (CI).

The term “pigments” should be understood to mean white or colored, mineral or organic particles, insoluble in an aqueous medium and intended for coloring the composition and/or the resulting deposit.

Preferably, the pigmentary dye of the invention has a refractive index of at least 1.9 at 25° C. The refractive index of the pigmentary dye of the invention is preferably comprised between 1.9 and 4. Especially, the pigmentary dye is different from fillers; preferably the pigmentary dye is different from talc.

The dyes may be present, in the composition, at a content ranging from 3% to 45% by weight, with respect to the weight of the composition, preferably from 4% to 30% by weight, preferably from 5% to 20% by weight, preferably from 6% to 15% by weight.

### Mineral Pigments

According to one particular embodiment, the pigments used according to the invention are selected from mineral pigments.

“Mineral pigment” means any pigment that complies with the definition in Ullmann's Encyclopedia in the Inorganic Pigment chapter. Mention can be made, among the mineral pigments useful in the present invention, of zirconium or cerium oxides, along with zinc, iron (black, yellow or red) or chromium oxides, manganese violet, ultramarine blue, chromium hydrate and iron blue, titanium dioxide, metallic powders such as aluminum powder and copper powder. The following mineral pigments can also be used: Ta2O5, Ti3O5, Ti2O3, TiO, ZrO2 in a mixture with TiO2, ZrO2, Nb2O5, CeO2, ZnS.

The size of the pigment useful in the context of the present invention is in general greater than 100 nm and may range up to 10 μm, preferably from 200 nm to 5 μm, and more preferably from 300 nm to 1 μm. According to a particular embodiment of the invention, the pigments have a size characterized by a D50 greater than 100 nm and may range up to 10 μm, preferably from 200 nm to 5 μm, and more preferably from 300 nm to 1 μm. The sizes are measured by static diffusion of light by means of a commercial MasterSizer 3000® granulometer from Malvern, making it possible to apprehend the granulometric distribution of all the particles over a wide range that can range from 0.01 μm to 1000 μm. The data are processed on the basis of the classic Mie scattering theory. This theory is the most adapted for size distributions ranging from submicron to multi-micron, and makes it possible to determine an “effective” diameter of particles. This theory is in particular described in the work by Van de Hulst, H. C., “Light Scattering by Small Particles”, Chapters 9 and 10, Wiley, New York, 1957. D50 represents the maximum size that 50% by volume of the particles have.

In the context of the present invention, the mineral pigments are more particularly iron oxide and/or titanium dioxide.

As mineral pigments that can be used in the invention, nacres can also be cited.

The term “nacres” should be understood to mean iridescent or non-iridescent colored particles of any shape, which are in particular produced by certain mollusks in their shell or else are synthesized and which exhibit a color effect by optical interference.

The nacres may be selected from pearlescent pigments such as titanium mica coated with iron oxide, titanium mica coated with bismuth oxychloride, titanium mica coated with chromium oxide, titanium mica coated with an organic dye, and pearlescent pigments based on bismuth oxychloride. This may also involve mica particles at the surface whereof are superposed at least two successive layers of metal oxides and/or of organic dyes. By way of example of nacres, mention may also be made of natural mica coated with titanium oxide, with iron oxide, with natural pigment or with bismuth oxychloride. The nacres may more particularly possess a yellow, pink, red, bronze, orange, brown and/or copper color or glint. Among the pigments that can be used according to the invention, mention can also be made of those with an optical effect different from a simple conventional hue effect, i.e. a unified and stabilized effect of the kind produced by conventional dyes, such as, for example, monochromatic pigments.

For the purpose of the invention, “stabilized” means absence of an effect of variability of color with the angle of observation or in response to a temperature change. For example, this material may be selected from particles having a metallic glint, goniochromatic coloring agents, diffracting pigments, thermochromatic agents, optical brighteners, and also fibers, in particular of the interference type. Of course, these various materials may be combined so as to provide the simultaneous manifestation of two effects, or even a new effect in accordance with the invention.

According to one particular embodiment, the composition according to the invention comprises at least one non-coated pigment.

### Organic Pigments

According to another embodiment of the invention, the pigmentary dye is an organic, synthetic or natural pigment or one of natural origin.

“Organic pigment” means any pigment that complies with the definition in Ullmann's Encyclopedia in the Organic Pigment chapter. The organic pigments can in particular be selected from the compounds nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane, quinophthalone.

The organic pigment or pigments can be selected for example from carmine, carbon black, aniline black, melanin, azo yellow, quinacridone, phthalocyanine blue, sorghum red, the blue pigments codified in the Color Index under the references CI 42090, 69800, 69825, 73000, 74100, 74160, the yellow pigments codified in the Color Index under the references CI 11680, 11710, 15985, 19140, 20040, 21100, 21108, 47000, 47005, the green pigments codified in the Color Index under the references CI 61565, 61570, 74260, the orange pigments codified the Color Index under the references CI 11725, 15510, 45370, 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, and the pigments obtained by oxidative polymerization of indole or phenolic derivatives as described in patent FR 2 679 771.

The pigments can also be in the form of composite pigments as described in patent EP 1 184 426. These composite pigments may be composed in particular of particles including an inorganic core covered at least partially with an organic pigment and at least one binder for fixing the organic pigments to the core.

The pigment may also be a lacquer.

The term lacquer means non-solubilized dyes adsorbed on insoluble particles, the assembly thus obtained remaining insoluble during use.

The inorganic substrates onto which the dyes are adsorbed are for example alumina, silica, calcium sodium borosilicate or calcium aluminum borosilicate, and aluminum.

Among the organic dyes, mention can be made of cochineal carmine. The products known under the following names can also be cited: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053), D&C Blue 1 (CI 42 090). By way of examples of lacquers, the product known by the name D&C Red 7 (CI 15 850:1) can be cited.

### Non-Ionic Surfactant

The composition according to the invention also comprises at least one non-ionic surfactant.

Preferably, the non-ionic surfactant used in the present invention has an HLB greater than or equal to 10, preferably greater than or equal to 12. As is well-known, HLB (Hydrophilic-Lipophilic Balance) means the balance between the size and strength of the hydrophilic group and the size and strength of the lipophilic group of the surfactant. The HLB value as per Griffin is defined in J. Soc. Cosm. Chem. 1954 (volume 5), pages 249-256.

Alternatively, preferably, the non-ionic surfactant used in the present invention has an HLB less than 10.

The non-ionic surfactant can be selected from the non-ionic surfactants known from the prior art, in particular from fatty acid and polyglycerol esters, sugar esters, poloxamers, polysorbates and mixtures thereof.

The non-ionic surfactant is preferably selected from fatty acid and polyglycerol esters and sugar esters.

The fatty acid and polyglycerol ester preferably comprises 2 to 10 glycerol units.

The fatty acid and polyglycerol ester is preferably formed from at least one acid comprising an alkyl or alkenyl chain, linear or branched, containing 10 to 20 carbon atoms and 2 to 10 glycerol units.

According to one embodiment, the polyglycerol ester according to the invention results from esterification of at least one saturated or unsaturated fatty acid and a polyglycerol comprising from 2 to 10 glycerol units.

Preferably, the fatty acid and polyglycerol ester is a mono- or diester, and preferably a mono-ester.

The term “polyglycerol” designates glyceryl polymers that are linear chains of 2 to 10, preferably 2, 3, 4, 5, 6 or 10 glycerol units.

The esters that are more particularly considered according to the present invention are esters resulting from the esterification of polyglycerol and C10-C20, preferably C10, C12 or C18 carboxylic acid(s), such as capric, lauric, ricinoleic, oleic, stearic, isostearic or myristic acids.

The carboxylic acid may be linear or branched, saturated or unsaturated.

Preferably, it is a linear monocarboxylic acid.

In general, they are derived from esterification of at least one hydroxyl function of a polyglycerol by a C10 to C20, preferably C12 to C18, and particularly C10 to C12 carbolic acid.

In one preferred embodiment of the invention, the fatty acid and polyglycerol ester is selected from polyglyceryl monolaurate comprising from 5 to 10 glycerol units, polyglyceryl monooleate comprising from 5 to 6 glycerol units, polyglyceryl mono(iso)stearate comprising from 5 to 6 glycerol units, polyglyceryl dioleate comprising from 5 to 6 glycerol units, polyglyceryl monomyristate comprising from 5 to 6 glycerol units, and mixtures thereof.

In a preferred embodiment of the invention, the ester is selected from the esters of fatty acid comprising 10 carbon atoms and of polyglycerol comprising 4 glycerol units, the esters of fatty acid comprising 12 carbon atoms and of polyglycerol comprising 10 glycerol units, and the esters of fatty acid comprising 18 carbon atoms and of polyglycerol comprising 2 glycerol units.

The ester of fatty acid comprising 10 carbon atoms and of polyglycerol comprising 4 glycerol units is formed (i) from at least one fatty acid comprising an alkyl or alkenyl chain containing 10 carbon atoms (also called C10 fatty acid), and (ii) from 4 glycerol units.

Preferably, the C10 fatty acid is saturated and contains a linear alkyl chain. Preferably, the C10 fatty acid is capric acid.

In a preferred embodiment of the invention, the ester of fatty acid comprising 10 carbon atoms and of polyglycerol comprising 4 glycerol units is polyglyceryl monocaprate comprising 4 glycerol units, i.e. polyglyceryl-4 monocaprate.

The esters of fatty acid comprising 12 carbon atoms and of polyglycerol comprising 10 glycerol units of is formed (i) from at least one fatty acid comprising an alkyl or alkenyl chain containing 12 carbon atoms (also called C12 fatty acid), and (ii) from 10 glycerol units.

Preferably, the C12 fatty acid is saturated and contains a linear alkyl chain. Preferably, the C12 fatty acid is lauric acid.

In a preferred embodiment of the invention, the ester of fatty acid comprising 12 carbon atoms and of polyglycerol comprising 10 glycerol units is polyglyceryl monolaurate comprising 10 glycerol units, i.e. polyglyceryl-10 monolaurate.

In another preferred embodiment of the invention, the composition comprises a fatty acid and polyglycerol ester that is a polyglyceryl monolaurate with 5 to 6 glycerol units, i.e. polyglyceryl-5 laurate or polyglyceryl-6 laurate.

The ester of fatty acid comprising 18 carbon atoms and of polyglycerol comprising 2 glycerol units is formed (i) from at least one fatty acid comprising an alkyl or alkenyl chain containing 18 carbon atoms (also called C18 fatty acid), and (ii) from 2 glycerol units.

Preferably, the C18 fatty acid is unsaturated and contains a linear alkyl chain. Preferably, the C18 fatty acid is oleic acid.

In a preferred embodiment of the invention, the ester of fatty acid comprising 18 carbon atoms and of polyglycerol comprising 2 glycerol units is polyglyceryl monooleate comprising 2 glycerol units, i.e. polyglyceryl-2 monooleate.

A commercial product mostly based on polyglyceryl-4 monocaprate or PG-4 caprate is available under the trade name TEGOSOFT PC 41 from the company Evonik Goldschmidt.

A commercial product mostly based on polyglyceryl-2 monooleate or PG-2 oleate is available under the trade name SUNSOFT Q-17D(G)-C from the company Taiyo Kagaku.

A commercial product mostly based on polyglyceryl-10 monolaurate or PG-10 laurate is available under the trade name Dermofeel G 10 L from the company Dr Straetmans.

A commercial product mostly based on polyglyceryl-5 laurate or PG-5 laurate is available under the trade name SUNSOFT A-121E-C® by the company Taiyo Kagaku.

A commercial product mostly based on polyglyceryl-6 laurate or PG-6 laurate is available under the trade name Dermofeel G 6 L by the company Dr Straetmans.

The fatty acid and polyglycerol ester may also be polyglyceryl-6 polyricinoleate or polyglyceryl-4 isostearate.

The composition according to the invention can also comprise at least one sugar ester. “Sugar ester” means a sugar and C10-C24 fatty acid ester, preferably C12-C20, and better still C12-C18. “Sugar” means compounds that have several alcohol functions with or without an aldehyde or ketone function and which include at least 4 carbon atoms. These sugars may be monosaccharides, oligosaccharides or polysaccharides.

As sugars that can be used according to the invention, mention can be made for example of sucrose (or saccharose), glucose, galactose, ribose, fucose, maltose, fructose, mannose, arabinose, xylose, lactose, and derivatives thereof, in particular alkylated, such as the methylated derivatives such as methyl glucose.

The fatty acids comprising from 10 to 24 carbon atoms may be linear or branched, saturated or unsaturated. The fatty acids can be selected from oleic acid, lauric acid, palmitic acid, myristic acid, stearic acid, linoleic acid, capric acid, behenic acid, cocoic acid, linolenic acid, capric acid, arachidonic acid, or mixtures thereof that lead in particular to mixed oleopalmitate, oleostearate and palmitostearate mixed esters or mixtures thereof. The esters can be selected from the mono-, di-, tri- and tetraesters, the polyesters and mixtures thereof.

By way of example, mention can be made of mono- and diesters and in particular mono- or dioleates, stearates, behenates, oleopalmitates, linoleates, linolenates, oleostearates, saccharose, glucose or methyl glucose, and in particular the product sold under the name Glucate DO by the company Amerchol, which is a methyl glucose dioleate.

Mention can also be made of the mixtures of methyl glucose and fatty acid esters such as the polyglyceryl-3 methylglucose distearate marketed by Evonik under the name Tego Care 450.

According to one particular embodiment of the invention, the sugar and fatty acid ester is a sucrose and fatty acid ester, preferably selected from the esters resulting from the reaction of sucrose(s) (saccharose) and fatty acid(s) comprising from 10 to 24 carbon atoms, preferably from 12 to 20 carbon atoms, better still from 12 to 18 carbon atoms.

In particular, the sucrose and fatty acid ester is selected from the esters resulting from the reaction of sucrose and fatty acid comprising from 12 to 18 carbon atoms such as lauric acid and/or palmitic acid, such as for example sucrose laurate, sucrose palmitate or a mixture.

The sucrose and fatty acid esters can be selected from the mono-, di-, tri- and tetraesters, the polyesters and mixtures thereof. Use is preferably made of esters with a low degree of esterification, such as for example sucrose and fatty acid monoesters, diesters or triesters or a mixture. The sucrose and fatty acid ester may be in the form of a mixture of esters with a low degree of esterification, such as for example a mixture of monoester and diester or a mixture of monoester, diester and triester.

In the case where use is made of a mixture of sucrose and fatty acid esters, a mixture is preferred in which the esters with a low degree of esterification, in particular monoesters, are in the majority and represent for example at least 50%, preferably at least 60% by weight of the mixture of sucrose and fatty acid esters.

By way of examples of esters or mixtures of sucrose and fatty acid esters, mention can be made of:
Surfhope SE COSME C-1803, which is a sucrose tristearate comprising approximately 20% monoester, the rest of the mixture being composed of di- and triesters,
Surfhope SE COSME C-1416, which is a sucrose myristate comprising approximately 80% monoester, the rest of the mixture being composed of di- and triesters,
Surfhope SE COSME C-1216, the INCI name of which is sucrose laurate, which comprises from 75 to 90% monoester, the rest of the mixture being composed of di- and triesters,
Surfhope SE COSME C-1215L, the INCI name of which is sucrose laurate, comprising approximately 70% monoesters, the rest of the mixture being composed of diesters and other polyesters,
Surfhope SE COSME C-1616, which is a mixture of saccharose esters and palmitic and/or stearic acids (INCI name sucrose palmitate), comprising from 75 to 90% monoester, the rest of the mixture being composed of di- and triesters, and may comprise sucrose stearate and sucrose palmitate stearate,
the ester bearing the INCI name sucrose laurate marketed by the company Daiichi Seiyaku under the reference DK ester S-L18A, comprising 70% monoesters and 30% di- and triesters,
the products sold under the names F50, F160, F140, F110, F90, F70 and SL40 by the company Crodesta, designating respectively the sucrose palmito-stearates formed by 30% monoester and 70% polyester, by 73% monoester and 27% di- and triester, by 61% monoester and 39% di-, tri- and tetraester, by 52% monoester and 48% di-, tri- and tetraester, by 45% monoester and 55% di-, tri- and tetraester, and by 39% monoester and 61% di-, tri- and tetraester, and sucrose monolaurate,
the products sold under the name RYOTO Sugar Esters for example referenced B370 and corresponding to the saccharose behenate formed by 20% monoester and 80% di-triester-polyester, or
the sucrose mono-di-palmito-stearate marketed by the company Goldschmidt under the name TEGOSOFT PSE.

Poloxamers that may be used in the invention are block copolymers of ethylene oxide and of propylene oxide of the following formula:

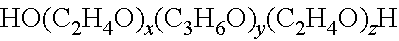

in which x, y, and z are integers such that x+z ranges from 2 to 100 and y ranges from 10 to 60, and mixtures thereof, and more particularly from the block copolymers with an HLB value ranging from 2 to 16.

Poloxamers can in particular be chosen from Poloxamer 184 (product of formula above with x=13, z=30, y=13); Poloxamer 231 (product of formula above with x=z=6, y=39; HLB 2); Poloxamer 282 (product of formula above with x=z=10, y=47; HLB 6); and Poloxamer 124 (product of formula above with x=z=11, y=21; HLB 16).

Polysorbates are sorbitan fatty esters. They can be chosen in particular from esters of a C—C fatty acid and of sorbitan and oxyethylenated esters of a C—C fatty acid and of sorbitan. They are formed from at least one fatty acid comprising at least one saturated linear alkyl chain, having, respectively, from 16 to 22 carbon atoms, and from sorbitol or from ethoxylated sorbitol. The oxyethylenated esters generally comprise from 1 to 100 ethylene oxide units, and preferably from 2 to 40 ethylene oxide (EO) units. These esters can be chosen in particular from stearates, behenates, arachidates, palmitates and mixtures thereof. Stearates and palmitates are preferably used. By way of example of sorbitan fatty ester and of an oxyethylenated sorbitan fatty ester, mention may be made of sorbitan monostearate (CTFA name: sorbitan stearate) sold by the company Croda under the name Span 60, sorbitan monopalmitate (CTFA name: sorbitan palmitate) sold under the name Span 40, sorbitan 20 EO monolaurate (CTFA name: polysorbate 20) sold under the name Tween 20, or sorbitan 20 EO tristearate (CTFA name: polysorbate 65) sold under the name Tween 65.

The non-ionic surfactant may be present in the composition according to the invention in a content ranging from 0.01% to 20% by weight, in relation to the total weight of the composition, preferably ranging from 0.02% to 10% by weight, preferably ranging from 0.03% to 8% by weight, preferably ranging from 0.05 to 5% by weight, preferably ranging from 0.08% to 4% by weight, preferentially ranging from 0.1% to 3% by weight, preferentially ranging from 0.5% to 2% by weight.

According to the invention, the weight ratio between the non-ionic surfactant selected from fatty acid and polyglycerol esters and sugar esters and chitosan (i.e. non-ionic surfactant to chitosan weight ratio) is between 0.02 and 10, preferably between 0.1 and 6. Preferably, it is between 0.1 and 5, preferably between 0.2 and 5, preferably between 0.2 and 4.5, preferably between 0.5 and 2.

### Physiologically Acceptable Aqueous Medium

The composition according to the invention comprises a physiologically acceptable aqueous medium. Said medium comprises water.

The water used can be sterile demineralized water and/or floral water such as rose water, cornflower water, chamomile water or linden water, and/or a spring or natural mineral water.

The composition preferably comprises at least 5% by weight water with respect to the total weight of the composition, preferably at least 10% by weight, preferably at least 20% by weight, preferably at least 30% by weight, preferably at least 40% by weight.

The composition preferably comprises from 5% to 95% by weight water with respect to the total weight of the composition, more preferentially from 10% to 85%, even more preferentially from 20% to 80%, even more preferentially from 25% to 70%, even more preferentially from 28% to 60%, even more preferentially from 30% to 50%.

The aqueous phase can also comprise at least one organic solvent miscible in water at 25° C.

Preferably, the water-miscible organic solvent is selected from alcohols, polyols and mixtures thereof.

Among the alcohols, mention can be made of C₁-C₁₀, more preferably C₁-C₅, alcohols, such as ethanol, isopropanol, propanol and butanol.

The polyol is, preferably, selected from polyols having from 2 to 20 carbon atoms, more preferentially from 2 to 6 carbon atoms, such as glycerol, diglycerol, propylene glycol, isoprene glycol, dipropylene glycol, butylene glycol, hexylene glycol, 1,2-propanediol, 1,3-propanediol, pentylene glycol, polyethylene glycols having from 2 to 200 ethylene oxide units and mixtures thereof.

The composition can comprise from 1% to 25% by weight water-miscible organic solvent, with respect to the total weight of the composition, more preferentially from 2% to 20% by weight, even more preferentially from 3% to 15% by weight.

### pH of the Composition

The composition according to the invention has a pH less than or equal to 7, preferably less than or equal to 6.5, preferably less than or equal to 6.3. Advantageously, the pH of the composition is between 3 and 6.3, preferably between 4 and 6.3.

Preferably, the cosmetic composition according to the invention comprises at least one base.

The base is particularly used to adjust the final pH of the composition to between 3 and 6.3.

The base can be selected from mineral bases such as for example alkali metal hydroxides, sodium hydroxide, potassium hydroxide.

Preferably, the base of the composition is an alkali metal hydroxide, preferably sodium hydroxide or potassium hydroxide. This is because these bases are more advantageous than nitrogenous bases, such as triethanolamine, on color stabilization (i.e. less yellowing).

The composition according to the invention can comprise at least one base at an active substance content ranging from 0.5% to 10% by weight, with respect to the total weight of the composition, particularly from 1% to 5% by weight, preferably ranging from 1% to 4% by weight.

The composition according to the invention can be obtained conventionally by those skilled in the art.

The following examples will make it possible to understand the invention better, without being in any way limitative. The raw materials are referred to by the chemical or INCI name thereof. The amounts indicated are as a % by weight of raw materials with respect to the total weight of the composition (% w/w), unless specified otherwise.

### EXAMPLES

### Preparation of Compositions

In the following examples, each formula was prepared in accordance with the protocol described below:

Under mechanical stirring with minimal vortex and at ambient temperature:
Disperse the chitosan and water under mechanical stirring with minimal vortex,
Add the appropriate quantity of acid to solubilize the chitosan and form a more viscous solution,
Add the pigments, then the polyols (if present), then the surfactants.
The mixture can then be passed through a triple-roll mill if necessary.

### Example 1: Evaluations of the Effects of the Various Ingredients

### 1. The Formulas F1 to F11 are Prepared.

Protocol Testing the Film-Forming and Peelability Aspect of the Formulas after Deposition

In an aluminum capsule 9.5 cm in diameter, deposit 10 g of solution and spread homogeneously over the entire surface. The deposit is left to dry for 24 hr at 32° C. on a hotplate. The sample is next evaluated to the touch initially. If the sample is liquid and remains stuck to the finger, it is considered that the sample is not capable of forming a film. In the case where the sample can form a film, a second evaluation consisting of detaching the sample from the aluminum capsule by means of a spatula. If the experimenter manages this, it will be said that the deposit is peelable.

The appearance is said to be compliant if the appearance of the preparation is fluid and can flow. The appearance will be non-compliant if the formulation is solid, or in the form of a gum or unable to flow.

The sensorial property is defined by spreading with a finger over a square of the skin of the arm 2.54 cm square. If the spreading is not fluid and clearly gives rise to sensations of irregularities of the bumpy texture or fragile solid type, it will be said that the sensorial property is non-compliant. If the spreading is fluid, it will be said that the sensorial property is compliant.

In the table: OK=compliant, NO=non-compliant, F5 and F6=Intermediate.

Homogeneity is described on the visual aspect of the preceding film. If it includes visible non-homogeneities of thickness, it will be said that the film is non-homogenous. If the film is visually of constant thickness, it will be said that the film is homogenous.

TABLE 1

| | | | | Appearance | | | | |
|---|---|---|---|---|---|---|---|---|
| | | | | of the | | | | Homogeneity |
| | Chitosan | AL | PG10L | composition | Film- | | Sensorial | of the film |
| F | (% w/w) | (% w/w) | (% w/w) | (bulk) | forming | Peelable | properties | on skin |
| F1 | 0.1 | 0.05 | | COMPLIANT | OK | NO | OK | |
| F2 | 0.5 | 0.25 | | COMPLIANT | OK | OK | OK | |
| F3 | 10 | 5 | | COMPLIANT | OK | OK | OK | |
| F4 | 15 | 7.5 | | NON- | OK | OK | NO since | Non- |
| | | | | COMPLIANT | | | solid | homogeneous |
| | | | | Elastic solid | | | | |
| F5 | 14 | 7 | | COMPLIANT | OK | OK | Exfoliates | Non- |
| | | | | | | | with | homogeneous |
| | | | | | | | difficulty | |
| | | | | | | | but OK | |
| F6 | 13 | 6.5 | | COMPLIANT | OK | OK | Exfoliates | Non- |
| | | | | | | | with | homogeneous |
| | | | | | | | difficulty | |
| | | | | | | | but OK | |
| F7 | 12 | 6 | | COMPLIANT | OK | OK | OK | homogeneous |
| F8 | 5 | 2.5 | 20 | COMPLIANT | OK | OK | OK | |
| F9 | 5 | 2.5 | 30 | NON- | OK | OK | OK | Homogeneous |
| | | | | COMPLIANT | | | | limit |
| | | | | limit | | | | |
| | | | | Elastic solid | | | | |
| F10 | 5 | 2.5 | 40 | NON- | OK | OK | OK | Non- |
| | | | | COMPLIANT | | | | homogeneous |
| | | | | Elastic solid | | | | |
| F11 | 5 | 2.5 | 50 | NON- | OK | OK | NO | Non- |
| | | | | COMPLIANT | | | | homogeneous |
| | | | | Elastic solid | | | | |

In the following table and hereinafter:
AL = lactic acid
PG10L = PG10 laurate

Formulas F1 and F2: The minimum dose of chitosan to form a film detectable by this test is 0.1%. The quantity of 0.5% makes it possible to obtain a peelable film.

Formula F4: Chitosan must in all cases be present in a quantity strictly less than 15% by weight, since the formula is then no longer applicable and the appearance of the composition (bulk) is no longer fluid.

Formulas F8 to F10: the weight ratio between non-ionic surfactant and chitosan is ideally less than or equal to 6 (F9), since beyond that the deposits are no longer homogeneous (F10), preferentially equal to 4 to obtain a very homogeneous film (F8). Non-homogeneity stems from the inability to work/mix/apply the material formed.

### 2. Protocol for Evaluating Modifications to the Mechanical Properties of the Films to Improve Comfort

The formulas C1 to C3 and A1, A3 and A4 are prepared.

In an aluminum capsule 9.5 cm in diameter, deposit 10 g of solution and spread homogeneously over the entire surface. The deposit is left to dry for 24 hr at 32° C. on a hotplate.
Cut 3 test pieces 20 mm long×6 mm wide using a cutting die.
Measure the thickness of the test pieces by means of a Palmer micrometer.
Place the test piece in the jaws of a TA.XT Plus texturometer from Stable Micro Systems and, using the Exponent software, initiate measurement in order to obtain the strength (g) and length at break (mm).
   TA Setting: speed 0.1 or 0.3 mm/s

$$\% deformation = elongation / initial size * 100 = ( elongation at break - initial size ) / initial size * 100$$

$$stress = strength ( N ) / surface ( m ) = strength ( g ) * g / width * thickness$$

TABLE 2

| | Chitosan | | | % Mean | | Mean | |
|---|---|---|---|---|---|---|---|
| | (100 kDa) | AL | PG10L | elongation | Error on | stress at | Error on |
| Formula | (% w/w) | (% w/w) | (% w/w) | at break | elongation % | break | stress |
| C1 | 5 | 2.5 | 0 | 4.8 | 0.9 | 46 | 7.5 |
| C2 | 5 | 2.5 | 1 | 60.8 | 7.8 | 17.5 | 3.0 |
| C3 | 5 | 2.5 | 10 | 259.3 | 9.6 | 4.2 | 0.8 |

These measurements make it possible to determine the minimum dose of non-ionic surfactant with respect to the chitosan. Having a softer film, and therefore one that has longer elongation at break, improves comfort on deposition.

Without surfactant, the chitosan film remains rigid and uncomfortable on the skin (C1).

The change in rigidity of the film is evaluated with the above measurements and shows that, from a weight ratio of non-ionic surfactant to chitosan equal to 1:5 (C2), the suppling effects are perceptible. This suppling is essential for comfort.

Formula A1 is comparative (marked by a star), while formulas A3 and A4 are according to the invention.

TABLE 9

| | | | | PG-3 | | | | | |
|---|---|---|---|---|---|---|---|---|---|
| | | | | methyl | | | | | |
| | Chitosan | | | glucose | Red | % Mean | Error on | Mean | |
| | (100 kDa) | AL | PG10L | distearate | pigment | elongation | elongation | stress at | Error on |
| Formula | (% w/w) | (% w/w) | (% w/w) | (% w/w) | (% w/w) | at break | % | break | stress |
| A1* | 5 | 2.5 | | | 15 | 1.3 | 0.1 | 18.2 | 1.2 |
| A3 | 5 | 2.5 | 10 | | 15 | 6.5 | 1.5 | 5.6 | 0.1 |
| A4 | 5 | 2.5 | | 10 | 15 | 6.5 | 0.5 | 0.2 | 0.1 |

Examples A3 and A4 show the impact of the surfactants on the flexibility of the film in the presence of pigment.

Formulas F, G, H and K according to the invention are prepared.

Mechanical properties are measured as indicated above.

TABLE 10

| | Chitosan | | | | | | Red |
|---|---|---|---|---|---|---|---|
| | (100 kDa) | AL | CA | Polox | PS20 | SucL | pigment |
| Formula | (% w/w) | (% w/w) | (% w/w) | (% w/w) | (% w/w) | (% w/w) | (% w/w) |
| F | 5 | 2.5 | | 10 | | | 15 |
| G | 5 | 2.5 | | | 10 | | 15 |
| H | 5 | 2.5 | | | | 10 | 15 |
| K | 5 | | 6 | | | 10 | 15 |

CA = citric acid
Polox = poloxamer 184 sold under the name SYNPERONIC PE/L 64-NAA-LQ-(CQ) by Croda
PS20 = polysorbate 20 sold under the name SP TWEEN 20 MBAL-LQ-(CQ) by Croda
sucL = sucrose laurate sold under the name SURFHOPE SE COSME C-1216 by MITSUBISHI-KAGAKU FOODS

The results are as follows:

TABLE 11

| | % Mean | | | |
|---|---|---|---|---|
| | elongation | Error on | Mean stress at | Error on |
| Formula | at break | elongation % | break (MPa) | stress (MPa) |
| F | 2.4 | 0.1 | 5.6 | 0.7 |
| G | 4.0 | 0.4 | 7.2 | 0.3 |
| H | 10.5 | 2.6 | 7.2 | 0.6 |
| K | 6.0 | 1.4 | 3.2 | 0.2 |

Examples F, G, H and K show the impact of the non-ionic surfactants such as poloxamers or polysorbates on the flexibility of the film in the presence of pigment. Example K further shows that citric acid (AHA) can be used instead of lactic acid.

3. Protocol for Evaluating Resistance to Rubbing when Dry

Some compositions are evaluated for their resistance to rubbing, by colorimetric measurements on dry film before and after abrasion, in accordance with the protocol detailed below.

### Protocol for Spreading Compositions in a Film

The products are spread on a spreading bench (Elcometer 4340 Applicator) making it possible to adjust its speed as well as the distance over which it takes place. The bench is equipped with an aspiration system connected to a pump so that the support where the spreading is done does not move. Contrast charts with non-varnished black background and white background are used (1 byko-chart, uncoated N2A, code 2831). As for the spreading thickness, this is adjustable by means of the square spreader deposited on the support so as to spread by levelling when the platform is started up. Each edge of the spreader makes it possible to spread with a different thickness ranging from 25 μm to 200 μm. The thickness selected is 50 μm in order to approach a thickness of the film in vivo. A weight of 960 g is added on top of the spreader during spreading. The spreading speed is adjusted at 1 in/sec, i.e. 2.54 cm/s. The films are dried for 24 hr at ambient temperature and humidity (50% RH).

TABLE 3

| | Chitosan | | | | |
|---|---|---|---|---|---|
| | (100 kDa) | AL | Pigments | PG10L | |
| Formula | (% w/w) | (% w/w) | (% w/w) | (% w/w) | Wetting |
| Comparative | 0.1 | 0.08 | 15 | 0 | No, there- |
| composition | | | | | fore non- |
| | | | | | homo- |
| | | | | | geneity |
| Composition | 0.1 | 0.08 | 15 | 0.1 | YES |
| according | | | | | |
| to the | | | | | |
| invention | | | | | |

The comparative composition cannot be spread to perform the contrast ratio (CR) tests that follow, it is said that it dewets. This dewetting causes non-homogeneities of deposition. This formula therefore does not meet the criteria for solving the technical problem of homogeneity and coverage because of dewetting.

On the other hand, the composition according to the invention has good wettability.

### Protocol for Measuring the Contrast Ratio (CR) to Objectify the Coverage

The color is measured by Konica Minolta CM-700d spectrophotometer. Measurement with contact guarantees the absence of light pollution.

Settings selected: Aperture 8 mm; Uncertainty: 0.04; SCI/SCE measurement; Geometry d/8°.

The color measurements on the two backgrounds (black background BB and white background WB) make it possible to characterize the coverage of a make-up foundation by calculating the contrast ratio (CR %) i.e. YBB/YWB×100, where YBB and YWB, where YBB and YWB are respectively the luminance values measured on black background and white background, this being higher, the more covering the make-up foundation.

Formula F12 is according to the invention, whereas formulas F13 to F16 are for comparison.

TABLE 4

| | Chitosan | | | | | |
|---|---|---|---|---|---|---|
| | (100 | | | | | |
| | kDa) | AL | Glycerin | Pigments | PG10L | |
| Formula | (% w/w) | (% w/w) | (% w/w) | (% w/w) | (% w/w) | CR |
| F12 | 1 | 0.5 | 1 | 3 | 0.1 | 52 ± 2 |
| (invention) | | | | | | 50 μm |
| F13 | 1 | 0.5 | 1 | 2.5 | 0.1 | 44 ± 1 |
| | | | | | | 50 μm |
| F14 | 1 | 0.5 | 1 | 2 | 0.1 | 43 ± 1 |
| | | | | | | 50 μm |
| F15 | 1 | 0.5 | 1 | 1.5 | 0.1 | 35 ± 2 |
| | | | | | | 50 μm |
| F16 | 1 | 0.5 | 1 | 1 | 0.1 | 19 ± 1 |
| | | | | | | 50 μm |

In order to achieve a minimum coverage fixed at 50%, it is necessary to have a minimum of 3% pigments.

TABLE 5

| | | Molecular | | | | | | |
|---|---|---|---|---|---|---|---|---|
| | Chitosan | weight of | | AL | Glycerin | Pigments | PG10L | |
| Formula | (% w/w) | chitosan | | (% w/w) | (% w/w) | (% w/w) | (% w/w) | CR 0 |
| F17 | 0 | | | | 5 | 15 | 1 | 84 ± 4 |
| F18 | 5 | <3 | kDa | | 5 | 15 | 1 | 89 ± 1 |
| F19 | 5 | 20 | kDa | 2.5 | 5 | 15 | 1 | 95 ± 1 |
| (invention) | | | | | | | | |
| F20 | 5 | 100 | kDa | 2.5 | 5 | 15 | 1 | 103 ± 3 |
| (invention) | | | | | | | | |
| F21 | 1 | 1 | MDa | 0.8 | | 15 | 0.1 | 100 ± 1 |
| (invention) | | | | | | | | |

Formulas F19 to F21 are according to the invention, whereas formulas F17 to F18 are for comparison.

Formulas F19 to F21 are the only ones that are significantly different from F17 (reference without chitosan) with regard to coverage in the deposit.

### Protocol for Testing Resistance to Rubbing

The test for resistance to rubbing is implemented by colorimetric measurements on dry film before and after abrasion. Abrasion is implemented by attaching a fabric handkerchief strip (Chicopee® Veraclean™ Polish Plus) on the spreader edge at 25 μm. The weight of 960 g is added on top of the spreader during abrasion. The bench speed is set at 2.54 cm/s.

The color measurement before and after abrasion is done with the same spectrophotometric method as explained before.

In order to evaluate resistance to rubbing, the contrast ratio respectively before rubbing (CR Dry Deposit, %) and after abrasion (CR Rub Deposit Dry, %) is measured.

The loss [(CR Rub Deposit Dry−CR Deposit Dry]/CR Deposit Dry]*100, as a percentage, quantifies the loss of coverage and indicates the resistance of the film to rubbing: the smaller this loss, the more the film is resistant to rubbing.

Each CR value in the following table therefore represents a mean of 6 measurements.

TABLE 6

| Formula | % Loss CR |
|---|---|
| F17 | 23.8 ± 2 |
| F18 | 26.3 ± 1 |
| F19 (invention) | 0 ± 1 |
| F20 (invention) | −0.3 ± 1 |
| F21 (invention) | −0.3 ± 1 |

The formulas F19 to F21 are the only ones not to lose coverage.

TABLE 7

| | Chitosan | | | | | |
|---|---|---|---|---|---|---|
| | (100 kDa) | AL | Glycerin | Pigments | PG10L | % Loss |
| Formula | (% w/w) | (% w/w) | (% w/w) | (% w/w) | (% w/w) | CR |
| F17 | 0 | | 5 | 15 | 1 | 23.8 ± 2 |
| F22 | 0 | | 0 | 15 | 0.1 | 21.9 ± 1 |
| F23 | 0.001 | 0.0008 | | 15 | 0.1 | 24.6 ± 2 |
| F24 | 0.005 | 0.004 | | 15 | 0.1 | 23.9 ± 1 |
| F25 | 0.01 | 0.008 | | 15 | 0.1 | 5.6 ± 1 |
| (invention) | | | | | | |
| F26 | 0.1 | 0.08 | | 15 | 0.1 | 0.4 ± 1 |
| (invention) | | | | | | |
| F27 | 5 | 4 | | 15 | 0.1 | 0.8 ± 1 |
| (invention) | | | | | | |
| F28 | 5 | 2.5 | 5 | 15 | 1 | −0.3 ± 1 |
| (invention) | | | | | | |

The formulas F25 to F28 are according to the invention, whereas formulas F22 to F24 are for comparison.

The formulas F25 to F28 are the only ones not to lose coverage (i.e. having a significantly smaller loss of coverage, i.e. a loss of no more than 6%).

### Test for Resistance to Oily Friction

The test consists in spreading 15 μL of formula on a disk 4 cm in diameter on the forearm and leaving the deposit to dry for 20 minutes. In parallel, 100 μL of oil (caprylic/capric triglyceride) is deposited on a fabric handkerchief strip (Chicopee® Veraclean™ Polish Plus). Next the handkerchief is rubbed with an axial force of 200-300 grams and the stain on the handkerchief is evaluated. The positive bound having a score of 5/5 corresponds to a non-stained tissue while, if the tissue is completely colored, a score of 1/5 will be attributed.

TABLE 8

| | Chitosan | | | | | |
|---|---|---|---|---|---|---|
| | (100 | | | | | Oil |
| | kDa) | AL | Pigments | PG10L | Dipropylene | strength |
| Formula | (% w/w) | (% w/w) | (% w/w) | (% w/w) | glycol | score |
| Comparative | 0 | | 15 | 1 | 12 | 1/5 |
| composition | | | | | | |
| Composition | 3 | 1.5 | 15 | 1 | 12 | 3/5 |
| according to | | | | | | |
| the | | | | | | |
| invention | | | | | | |

Under these conditions the composition according to the invention performs so as to obtain a score of 3/5. It can be seen that chitosan improves the resistance to oily friction of the deposits.

### Example 2: Comparative Data

A composition similar to composition of Example 11 (Emulsion foundation) of JP-H10-265337, called HI* (out of invention), was prepared as follows (% w/w):
PG-10 laurate 9.00
Purified water Qsp 100
Red pigments 10.7
Lactic acid 0.15
Chitosan (molecular weight 100 kDa) 0.01 (% active material)

The weight ratio between non-ionic surfactant and chitosan for said composition is 9/0.01=900.

The same composition but comprising 1% by weight of chitosan was prepared (invention; weight ratio between non-ionic surfactant and chitosan=1/1=1).

Resistance to rubbing was measured as detailed in Example 1. The results are as follows:

TABLE 9

| | Chitosan | | | Red | |
|---|---|---|---|---|---|
| | (100 kDa) | AL | PG10L | pigment | |
| | (% p/p) | (% p/p) | (% p/p) | (% p/p) | % Loss CR |
| HI* | 0.01 | 0.15 | 9 | 10.7 | 56.6 +/− 18.7 |
| Invention | 1 | 0.5 | 1 | 10.7 | 0.13 +/− 0.1 |

This shows that the claimed weight ratio between non-ionic surfactant and chitosan allows obtaining a significantly increased resistance to rubbing as compared to prior art.

1. A cosmetic composition comprising, in a physiologically acceptable aqueous medium:
a) at least 0.01% by weight, as a proportion of the total weight of the composition, native chitosan having a molecular weight strictly greater than 3000 daltons, this quantity being strictly less than 15% by weight,
b) at least one alpha hydroxy acid,
c) from 3% to 50% pigmentary coloring material with respect to the total weight of the composition, and
d) a non-ionic surfactant,
wherein the weight ratio between non-ionic surfactant and chitosan is between 0.02 and 10.

2. The composition according to claim 1, which comprises from 0.01% to 14% by weight of the native chitosan with respect to the total weight of the composition.

3. The composition according to claim 1, wherein the native chitosan has a molecular weight greater than or equal to 10 kDa; and/or the degree of acetylation of the chitosan is less than or equal to 50%.

4. The composition according to claim 1, wherein the chitosan is extracted and purified from food or biotechnological fungal sources.

5. The composition according to claim 1, wherein the α-hydroxy acid is selected from lactic acid, citric acid, methyllactic acid, glucuronic acid, glycolic acid, pyruvic acid, 2-hydroxy-butanoic acid, 2-hydroxypentanoic acid, 2-hydroxyhexanoic acid, 2-hydroxyheptanoic acid, 2-hydroxyoctanoic acid, 2-hydroxy-nonanoic acid, 2-hydroxydecanoic acid, 2-hydroxyundecanoic acid, 2-hydroxydodecanoic acid, 2-hydroxytetradecanoic acid, 2-hydroxy-hexadecanoic acid, 2-hydroxyoctadecanoic acid, 2-hydroxytetra-cosanoic acid, 2-hydroxyeicosanoic acid; mandelic acid; phenyllactic acid; gluconic acid; galacturonic acid; aleuritic acid; ribonic acid; tartronic acid; tartaric acid; malic acid; fumaric acid; salts and mixtures thereof.

6. The composition according to claim 1, wherein the alpha hydroxy acid is present in a quantity ranging from 0.001 to 10% by weight with respect to the total weight of the composition.

7. The composition according to claim 1, wherein the dye is selected from powder dyes.

8. The composition according to claim 1, wherein the non-ionic surfactant is selected from fatty acid and polyglycerol esters and sugar esters.

9. The composition according to claim 1, wherein the non-ionic surfactant is selected from the esters resulting from the esterification of polyglycerol and C10-C20.

10. The composition according to claim 1, wherein the non-ionic surfactant is selected from sugar and C10-C24 fatty acid esters.

11. The composition according to claim 1, wherein the non-ionic surfactant is present in a content ranging from 0.01% to 20% by weight, in relation to the total weight of the composition.

12. The composition according to claim 1, wherein the weight ratio between non-ionic surfactant and chitosan is between 0.1 and 5.

13. The composition according to claim 1, which comprises at least 30% by weight water with respect to the total weight of the composition; and optionally at least one organic solvent miscible in water.

14. The composition according to claim 1, which has a pH less than or equal to 7.

15. A method for making up and/or caring for the skin and/or the keratin appendages, wherein the composition according to claim 1 is applied to the skin and/or the keratin appendages.

16. The composition according to claim 1, wherein the weight ratio between non-ionic surfactant and chitosan is between 0.1 and 6.

17. The composition according to claim 1, wherein the alpha hydroxy acid is selected from lactic acid, citric acid and the salts and mixtures thereof.

18. The composition according to claim 1, wherein the dye is selected from iron oxide, titanium dioxide and mixtures thereof.

19. The composition according to claim 1, wherein the non-ionic surfactant is selected from mixtures of fatty acid esters and methyl glucose.

20. The composition according to claim 1, wherein the weight ratio between non-ionic surfactant and chitosan is between 0.2 and 5.
